(19) United States
(12) Patent Application Publication
(10) Pub. No.: US 2003/0147773 A1
(43) Pub. Date: Aug. 7, 2003

(54) Apparatus and method for paper and dry goods bio-decontamination

(76) Inventors: Roger S. Bryner, Salt Lake County, UT (US); Neil Arnold, Salt Lake County, UT (US)

Correspondence Address: Roger S Bryner, 6751 South 2300 East, Salt Lake City, UT, 84121, US

(21) Appl. No.: 10/061,534

(22) Filed: Feb. 1, 2002

(51) Int. Cl. A61L 9/00; G01D 11/26; B01D 11/02; A61L 2/00

(52) U.S. Cl. 422/28; 422/1; 422/5; 422/32; 422/119; 422/261; 422/292; 422/305

(57) ABSTRACT

The present invention is an apparatus and method for the sanitation of biologically contaminated dry goods using a combined gaseous chemical and thermal treatment. The invention consists of three primary items: (1) an chemical dispenser, (2) a sanitizing chamber, and (3) a method of decontamination employing the previous two items. The dispenser is designed to eliminate the need for direct operator handling of the sanitizing chemicals, while providing for the release of an appropriate volume of chemical into the gas phase for the treatment process at the desired treatment temperature. The treatment chamber provides for containment of the chemical treatment gases at the desired concentration for the duration of the treatment. The method involves the use of a mixture of the chemicals used in combination with the dispenser and chamber. The chemical mixture is selected for chemical stability during storage in the dispenser and efficiency during treatment.

### CROSS REFERENCES TO RELATED APPLICATIONS

6,228,330 May 8, 2001 Herrmann et al 422/186.05

4,861,956 Aug. 29, 1989 Courneya 219/746

5,772,971 Jun. 30, 1998 Murphy et al 422/292

5,744,688 Apr. 28, 1998 Vance et al, 588/205

5,958,336 Sep. 28, 1999 Duarte 422/24

5,894,014 Apr. 13, 1999 Huston et al 422/295

5,355,901 Oct. 18, 1984 Milenik et al 134/105

5,217,698 Jun. 8, 1993 Siegel et al 422/295

4,863,688 Sep. 5, 1989 Schmidt et al 422/28

1,663,841 Mar. 1, 1928 Hollenbeck 422/112

6,193,931 Feb. 27, 2001 Lin et al 422/28

5,843,375 Dec. 1, 1998 Emerson, et al 422/36

### ADDITIONAL REFERENCE

Pickerill J. K., “Practical System for Steam-Formaldehyde Sterilizing”; Lab. Pract. (GB), vol2 24, No. 6, 6/75; pp. 401-404

### Background-Field of Invention

This invention describes an apparatus and method for the sanitation of biologically contaminated dry goods using a combined gaseous chemical and thermal treatment.

### BACKGROUND-DESCRIPTION OF PRIOR ART

Various combinations of heat and chemical treatments have been previously utilized for the decontamination and sterilization of materials exposed to infectious biological agents. Most common are a variety of solution and solvent based “wet” chemical treatments. These techniques work well on a variety of non-porous surfaces and materials that are non-reactive to the cleaning agent. Unfortunately materials that are easily damaged by water, easily damaged by the high concentrations of chemicals associated with such techniques or highly porous are not suitable to such treatments. Further the potential for worker exposure to the infectious agent is high during such cleaning procedures.

Methods for “dry” decontamination typically involve a greater complexity and may still damage the materials being processed. Two methods are most common: (1) is the direct application of heat at temperatures above 350 degrees Fahrenheit in an air or other oxygen rich atmosphere, and (2) is the use of steam (gaseous H2O) at 2 atmospheres and 250 degrees Fahrenheit.

The first of these methods combines extreme heat with the ubiquitous presence of oxygen to destroy any infectious agents. Unfortunately few dry goods items can survive the extreme nature of this treatment without damage such as browning, melting or even catching on fire. The second method typically involves an expensive and unwieldy autoclave for pressure treatment. This device is very effective, but does present additional hazards of a high pressure device that may fail catastrophically if it is not maintained and operated properly.

A variety of additional variations on these dry decontamination methods exist. They use different chemical agents such as chlorine dioxide, formaldehyde, nitrogen tetroxide, methyl bromide, methanol which can be extremely hazardous if present in sufficient quantity. These hazardous conditions can result from toxicology, corrosivity or explosivity. These chemical approaches typically involve some addition of heat or stream to be effective. The use of chlorine dioxide combined with water vapor at room temperature, however, has also been attempted. This method failed on the first application to remove all traces of anthrax from contaminated office buildings and required extreme containment measures.

In every case, establishing the proper temperatures and concentrations for treatment of material is difficult, and yet it is extraordinarily import if the sterilization process is to be effective.

### SUMMARY INCLUDING OBJECTS AND ADVANTAGES

The present invention is an apparatus and method for the sanitation of biologically contaminated dry goods using a combined gaseous chemical and thermal treatment. This invention provides a means for chemical decontamination of dry goods that does not require an expressive pressure containment apparatus or extensive chemical handling facilities. This invention is suitable for the decontamination of a wide variety of materials, such as mail, paper, plastics, fabrics, clothes and wood items. Many of these items are completely incompatible with common solution based decontamination methods and high temperature direct thermal treatments. Further, this invention provides a simple, safe and effective means for decontamination that can readily be applied outside of an industrial or laboratory environment.

The invention consists of three primary items: (1) an chemical dispenser, (2) a sanitizing chamber, and (3) a method of decontamination employing the previous two items. The dispenser is designed to eliminate the need for direct operator handling of the sanitizing chemicals, while providing for the release of an appropriate volume of chemical into the gas phase for the treatment process at the desired treatment temperature. The treatment chamber provides for containment of the chemical treatment gases at the desired concentration for the duration of the treatment. The method involves the use of a mixture of the chemicals (water, methanol, methanal and methanoic acid) used in combination with the dispenser and chamber. The chemical mixture is selected for chemical stability during storage in the dispenser and efficiency during treatment. The primary active agent is believed to be methanal.

### OBJECTS AND ADVANTAGES

Accordingly, several objects and advantages of this invention are realized when compared to existing sterilization techniques. The methods and apparatus provide a “dry” gas phase decontamination method compatible with a wide variety of dry goods. While existing methods require expensive and potentially dangerous pressure vessels, this method uses cost effective containment of gas only without pressure. In addition, no solid or liquid waste stream is produced by this method reliving the necessity of drainage and venting. The use of readily available materials coupled with these advantages result in a much lower cost. Additionally the method itself has advantages over existing methods. Heat alone requires temperatures high enough to badly scorch and burn dry goods such as paper, while this method does not result in browning of paper. Wet methods are sometimes incompatible with dry goods which must be carefully dried after steam autoclaving. The combination of treatment container and pre-measured dose package results in a correct exposure and containment at safe exposure levels in all uses without exposure to the treatment chemicals.

Still further objects and advantages will become apparent from a consideration of the ensuing description and accompanying drawings.

### DESCRIPTION-MAIN EMBODIMENT

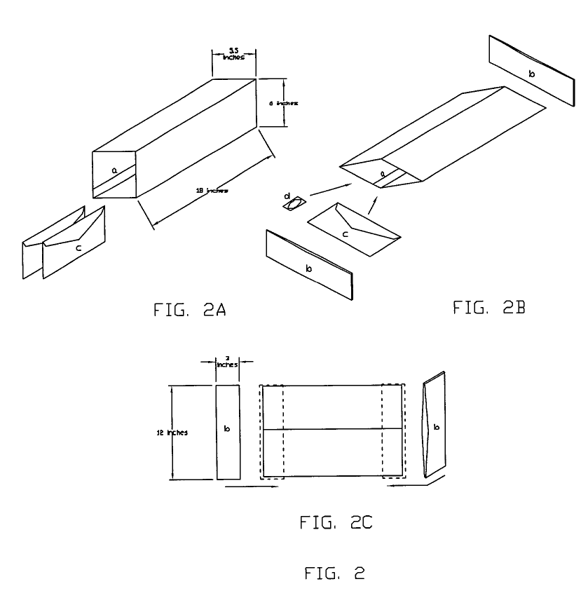

As can be seen in FIG. 2B item d, a sealed pouch is used to hold the liquid or solid phase fumigant sometimes in conjunction with an absorbent or supportive inert carrier. This pouch is composed of a material such as saran, polyethylene, or other thermoplastic that fails upon heating and the pressure exerted by the gaseous fumigant. Alternatively the pouch may be composed mostly of a completely impermeable material, such as aluminum foil with an area of failing plastic or adhesive to act as a release valve. This sealed container can also be used for shipping and under normal shipping conditions up to 135 degrees Fahrenheit will not fail. This package will also have some method of indicating that the agent is present in the package, both after shipping and in the negative after treatment.

The main function of the treatment tube is to hold the gaseous fumigants in contact with the dry goods in a controlled environmental condition. The tube can include heating elements and sensing elements in industrial applications, however can be as simple as a foil coated cardboard tube for economical applications. A novel feature of the tube shown is that when configured to a square shape, it lines the inside of a normal sized mail box closely with a volume of over 10 Liters. When folded flat, it reduces it's volume by over ⅕ and seals in an expedient manner, resulting in efficient and nonhazardous use of the fumigant agent. Larger volumes require ventilation or reabsorbing of the active agent to comply with now current OSHA guidelines for exposure to the active ingredients.

The method for sterilization of dry goods relies on a heated containment chamber filled with an active fumigant, such as a mixture of methanal, methanol, and methanoic acid with water vapor. Such a mixture, when exposed to dry goods, kills spore forming bacteria and other biological agents by acting on the proteins of living organisms. The gasses involved permeate all but sealed foil or other such sealed containers. While it is possible that material inside such a container could be contaminated and mailed, it is much more likely that cross contamination from mailed goods would result in large volumes of mail contaminated with invisible specs of lethal powder in the case of an Anthrax attack through the mail. In this area this treatment is less effective(but more economical) than radiation based treatments. No chemical or liquid treatment is inherently more advantageous that this treatment.

### PREFERRED EMBODIMENT-OPERATION

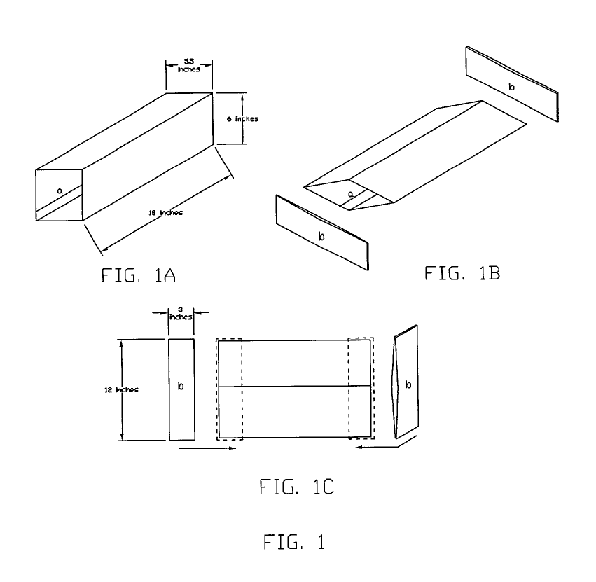

The preferred operation of this system is illustrated by FIG. 1A The tube is placed in its open square form inside a standard mail box. Once the mail is delivered, the sealed pouch is placed inside the tube with the mail and the whole assembly collapsed to reduce volume as shown in FIG. 2B. Once the tube is collapsed the end sleeves or clamps are placed to hold the tube closed as shown in FIG. 2C. The sleeve is then heated to between 160 and 220 degrees Fahrenheit. As the mail heats up the sealed pouch also heats and releases its fumigant payload when an effective temperature is reached. Indication of release and subsequent drying of the carrier is performed by standard indicators in solution and on the carrier in the sealed pouch. With time and temperature the dry goods are sterilized without harming them with excessive heat. Once the prescribed treatment time is reached the tube is opened and ventilated outside or in a window. The contents are then retrieved from inside and used normally.

### CONCLUSIONS

Accordingly, it can be seen that the apparatus and method described significantly differs from prior art by combining novel elements to produce a decontamination apparatus and method which does not use pressure, does not use extreme heat, does not use oxygen and heat, does not use high pressure steam, does not use wet solution or solvent based treatment, does not require direct operator contract with treatment chemicals, does not require operator contact with potentially contaminated dry goods, does not damage temperature or moisture sensitive materials, does not require special drying after treatment, does not produce a hazardous liquid or solid waste stream, and does not require an industrial treatment facility.

Instead the invention uses a unique chemical dispensing system capable of releasing the active agents to the gas phase once the process has reached the effective temperarure. It uses a simple, low cost treatment vessel which controls chemical release, maintains the chemical concentration and prevents operator exposure to treatment chemicals. Provides a treatment method which employs a combination of temperature and gas phase concentration effective for decontamination of dry goods. As such, this apparatus and method is unique and deserving of patent protection.

What is claimed is:

1) An sealed container for safe and stable containment of a fumigant agent during storage, handling and shipping which comprises in combination.
a) An auto release mechanism for dispensing the fumigant from the sealed container
b) A means to indicate that the contents have been successfully dispensed
c) A means to indicate that the contents have stored successfully without being leaked

2) A container defined in claim 1 also incorporating a thermally activated auto release mechanism for the sealed container

3) A container defined in claim 1 also incorporating an absorbent material to hold and immobilize liquid phase fumigants during release and is inert under storage and release of the fumigant

4) A container defined in claim 1 that includes a release by failure area consisting of a material that is impermeable in shipping but that develops permeability and failure during treatment

5) A treatment container for fumagation which comprises in combination
a) that can act as a holding/heating container for the sealed container defined under claim 1
c) that is Stable to heat and fumigant exposure during treatment
d) that is impermeable to fumigants during treatment and maintains the proper concentration profile

6) A treatment container defined in claim 5 that acts as both a postal mail receiving container and a treatment container

7) A treatment container defined in claim 5 that can reduce it's volume after mail is delivered but before it is treated

8) A treatment container defined in claim 5 that configures to line the inside walls of standard mail boxes.

9) A treatment container defined in claim 5 that includes a facility to alone or in combination reabsorb, neutralize, or vent the fumigant after treatment

10) An implementation of the treatment container defined in claim 5 that includes heating elements thus not requiring an external oven.

11) A method to sterilize mail, currency, printed goods, and dry goods which comprises in combination.
a) a method using a controlled chemical and thermal environment.
b) using a controlled release of fumigant
c) using a treatment container such as that defined in claim 5,6,7,8,9 or 10
d) Using a mixture of chemical active agents augmented by water vapor
f) A method to control concentration or both active and side components of the fumigation mixture
g) A method to guarantee temperature/treatment profiles of bulk quantities of dry goods, mail, currency, and printed materials
h) A method to insure safe handling of dry goods, mail, and currency prior to treatment.

12) A treatment method defined in claim 11 using an automatically controlled release of fumigant

13) A treatment method defined in claim 11 using a temperature controlled release of fumigant

14) A treatment method defined in claim 11 using a sealed container such as that defined in claims 1,2,3 or 4

15) A treatment method defined in claim 11 using an activation of a solid or liquid compound to produce a release of gas phase fumigant

16) A treatment method defined in claim 11 using thermal activation of a solid or liquid compound to produce methanal gas

17) A treatment method defined in claim 11 using Thermal activation of a solid or liquid compound to produce a mixture of methanal and methanoic acid

18) A treatment method defined in claim 11 using thermal activation of a solid or liquid compound to produce a mixture of methanol, methanol and methanoic acid
